(19) United States
(12) Patent Application Publication
(10) Pub. No.: US 2023/0233798 A1
(43) Pub. Date: Jul. 27, 2023

(54) Catheter Insertion System

(71) Applicant: Bard Access Systems, Inc., Salt Lake City, UT (US)

(72) Inventors: BreAnna E. Lackey, West Valley City, UT (US); Yiping Ma, Layton, UT (US)

(21) Appl. No.: 18/098,632

(22) Filed: Jan. 18, 2023

Related U.S. Application Data
(60) Provisional application No. 63/302,774, filed on Jan. 25, 2022.

(51) Int. Cl. A61M 25/00; A61M 25/01

(52) U.S. Cl. CPC A61M 25/0043; A61M 25/0127; A61M 2025/0059; A61M 2025/0166

(57) ABSTRACT

A catheter assembly including a multi-luminal catheter, a primary stylet, and a secondary stylet. The primary stylet and the secondary stylet are each inserted into separate lumens extending along the catheter tube. The primary stylet includes a magnetic region to enable magnetic tracking. The secondary stylet is configured to prevent buckling of the magnetic region during insertion. The secondary stylet includes a proximal section having a proximal column strength and a distal section having a distal column strength greater than the proximal column strength. During use, an insertion force exceeding an insertion force limit causes the proximal section to buckle to prevent buckling of the distal section. The primary stylet may also include electrical sensors and/or an optical fiber configured for shape sensing.

### PRIORITY

This application claims the benefit of priority to U.S. Provisional Application No. 63/302,774, filed Jan. 25, 2022, which is incorporated by reference in its entirety into this application.

### BACKGROUND

Inserting a peripherally inserted central catheter (“PICC”) into the vasculature of the patient may involve a stylet being placed inside a lumen of the PICC to give the PICC stabilization during insertion of a PICC-stylet assembly. However, the tip of the PICC can encounter obstructions during placement, leading to buckling or a bending event of the tips of the stylet and the PICC. The bending event can lead to breaking off of the tip of the stylet, wherein breaking off of one or more of the tip is considered a serious failure of the PICC and can lead to adverse outcomes for the patient. It would be beneficial to the patient and the clinician to have a catheter insertion system that are not prone to bending events, allowing the clinician to properly place the PICC without fear of PICC or stylet failure. Disclosed herein are a catheter assembly, system and method of use that address the foregoing.

### SUMMARY

Disclosed herein is a catheter assembly that, according to some embodiments, includes, a multi luminal catheter having a first extension leg in fluid communication with a first lumen and a second extension leg in fluid communication with a second lumen. A primary stylet is inserted within the first lumen, where the primary stylet includes a magnetic region configured to enable a medical tracking system to track the primary stylet during advancement of the catheter along a vasculature of a patient and a secondary stylet inserted within the second lumen. The distal section of the secondary stylet is configured to inhibit buckling of the magnetic region of the primary stylet during advancement of the catheter assembly along a vasculature of a patient.

In some embodiments, the distal section defines a distal column strength greater than a column strength of the magnetic region of the primary stylet, and in some embodiments, the distal section extends along the magnetic region.

In some embodiments, a proximal section of the secondary stylet defines a proximal column strength, where the proximal column strength is less than the distal column strength. In some embodiments, the diameter of the proximal section is less than the diameter of the distal section and in some embodiments, the secondary stylet defines a transitioning section extending between the proximal section and the distal section, where the transitioning section defines a diameter between distal diameter and the proximal diameter. In some embodiments, the transitioning section defines a taper extending along the transitioning section, where the taper defines the proximal diameter at the proximal end of the transitioning section and the distal diameter at the distal end of the transitioning section.

In some embodiments, the proximal section extends proximally beyond the second extension leg such that the proximal section is configured to receive a catheter insertion force applied thereto by a clinician.

In some embodiments, the proximal column strength defines an insertion force limit for the catheter, when the insertion force is applied to the secondary stylet, and in some embodiments, the proximal section is configured to buckle when the insertion force exceeds the insertion force limit. In some embodiments, the insertion force limit is configured to prevent buckling of the magnetic region.

In some embodiments, the primary stylet includes a sheath extending along the magnetic region, and the magnetic region includes one or more magnets disposed within the sheath.

In some embodiments, the primary stylet includes one or more sensors configured to detect one or more electrical signals. In some embodiments, the one or more sensors include an electrode configured to detect an ECG signal so as to enable the medical tracking system to confirm a location of the catheter within the vasculature. In some embodiments, the primary stylet includes an optical fiber extending therealong, where the optical fiber is configured to enable shape sensing of the primary stylet by the medical tracking system.

In some embodiments, the secondary stylet includes at least one of the magnetic region, the one or more electrical sensors, or the optical fiber.

In some embodiments, the primary stylet includes a primary stylet attachment device configured to selectively couple the primary stylet to the first extension leg such that longitudinal displacement of the primary stylet with respect to the catheter is inhibited. In some embodiments, the secondary stylet includes a secondary stylet attachment device configured to selectively couple the secondary stylet to the second extension leg such that longitudinal displacement of the secondary stylet with respect to the catheter is inhibited.

Also disclosed herein is a method for placing a catheter within a vasculature of a patient that, according to some embodiments, includes providing a catheter assembly that includes a multi-luminal catheter having a first extension leg in fluid communication with a first lumen and a second extension leg in fluid communication with a second lumen. The catheter assembly further includes a primary stylet inserted within the first lumen, where the primary stylet includes a magnetic region configured to enable a medical tracking system to track of the primary stylet during advancement of the catheter along a vasculature of a patient. The catheter assembly further includes a secondary stylet inserted within the second lumen, where the secondary stylet defines a proximal section extending proximally beyond the second extension leg. The method further includes applying an insertion force to the proximal section to advance the catheter along the vasculature.

In some embodiments of the method, the secondary stylet defines a distal section having a distal column strength greater than a column strength of the magnetic region of the primary stylet. In some embodiments of the method, the proximal section includes a proximal column strength that is less than the distal column strength.

In some embodiments of the method, providing the catheter assembly includes (I) inserting the primary stylet into the first lumen and (ii) inserting a secondary stylet into the second lumen such that the distal section is disposed adjacent the magnetic region.

In some embodiments of the method, the proximal column strength defines an insertion force limit for the catheter, such that the proximal section is configured to buckle when the insertion force applied by the clinician exceeds the insertion force limit. In some embodiments of the method, the insertion force limit prevents buckling of the magnetic region.

In some embodiments, the method further includes coupling a primary stylet attachment device between the primary stylet and a first extension leg of the catheter to inhibit longitudinal displacement of the primary stylet with respect to the catheter. In some embodiments, the method further includes coupling a secondary stylet attachment device between the secondary stylet and a second extension leg of the catheter to inhibit longitudinal displacement of the secondary stylet with respect to the catheter.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of a catheter insertion system, in accordance with some embodiments;

FIGS. 2A-2B illustrates a plan view of some components of the catheter insertion system including a primary stylet and a secondary stylet;

FIG. 2C illustrates a cross sectional view of the primary stylet and the secondary stylet placed inside the catheter, in accordance with some embodiments;

FIGS. 3A-3C illustrate various views of the system of FIG. 1 depicting an exemplary method of inserting a catheter, in accordance with some embodiments;

FIGS. 3D-3E illustrate views of the system of FIG. 1 inserted with within a blood vessel having an obstruction therein, in accordance with some embodiments; and

FIG. 4 illustrates a flow chart of an exemplary method of placing a catheter, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

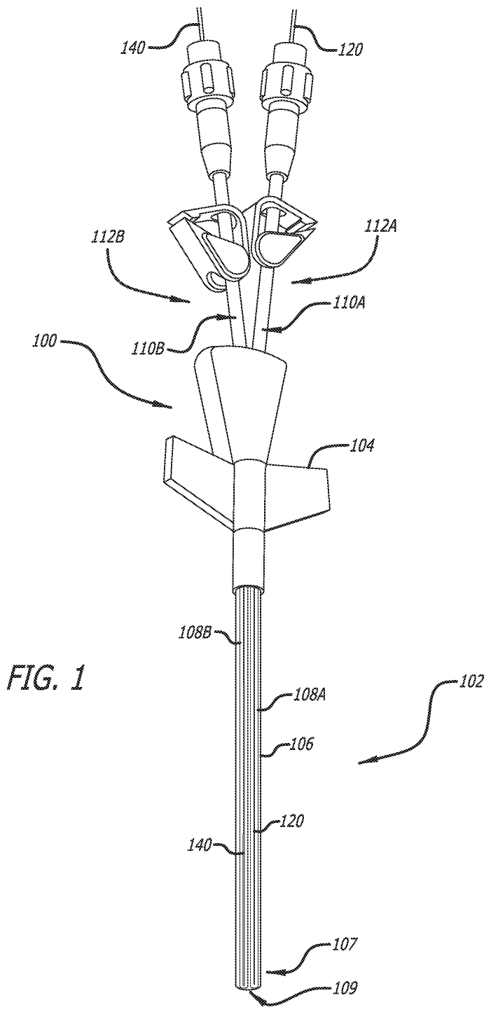

FIG. 1 illustrates a perspective view of a catheter insertion system (or catheter assembly) 100, in accordance with some embodiments. The catheter insertion system (“system”) 100 includes a multi-luminal catheter 102, e.g., a peripherally inserted central catheter (“PICC”). The catheter 102 includes a hub 104 distally coupled to a catheter tube 106. The catheter tube 106 includes a first lumen 108A and a second lumen 108B extending along the catheter tube 106 between the hub 104 and a distal opening 109 at a distal end 107 of the catheter tube 106. The catheter 102 includes a first extension leg 110A and a second extension leg 110B coupled with the hub 104. The first extension leg 110A includes a first extension leg lumen 112A in fluid communication with the first lumen 108A, and the second extension leg 110B includes a second extension leg lumen 112B in fluid communication with the second lumen 108B. For ease of explanation, embodiments of the system 100 are described in terms of a dual lumen catheter. However, it will be appreciated that in some embodiments, the system 100 may be designed to include catheters having more than two lumens which are considered to fall within the scope of the present invention.

The system 100 further includes a primary stylet 120 and a secondary stylet 140. The primary stylet 120 and the secondary stylet 140 are inserted into the catheter 102. More specifically, the primary stylet 120 is inserted into the first extension leg lumen 112A and the first lumen 108A. Similarly, the secondary stylet 140 is inserted into the second extension leg lumen 112B and the second lumen 108B. In some embodiments, the catheter 102 may be provided having one or both of the primary stylet 120 and the secondary stylet 140 pre-inserted. In other embodiments, a clinician may insert either or both of the primary stylet 120 and the secondary stylet 140 at the time of or during placement of the catheter 102 into a vasculature of a patient.

In some instances of catheter advancement along the vasculature, the catheter may encounter resistance. For example, a distal tip of the catheter may abut an obstruction (e.g., a blood vessel wall) during advancement. In such instances, the catheter (e.g., a distal end portion) may buckle and in some instances, the buckling may cause damage to the primary stylet 120 and/or the catheter 102. By way of further example, the catheter 102 having the primary stylet 120 inserted therein, may abut an obstruction during advancement causing a distal end portion of the catheter 102 and the primary stylet 120 to buckle. In some instances, the bucking of the primary stylet 120 may cause damage to the primary stylet 120. The secondary stylet 140 is generally configured to inhibit/prevent buckling of the primary stylet 120 in such instances. In some instances, the buckling of the primary stylet 120 may cause a distal portion of the primary stylet 120 to break rendering the primary stylet 120 non-functional. As such, the secondary stylet 140, when inserted and utilized during advancement of the catheter 102, may protect the primary stylet 120 against loss of functionality.

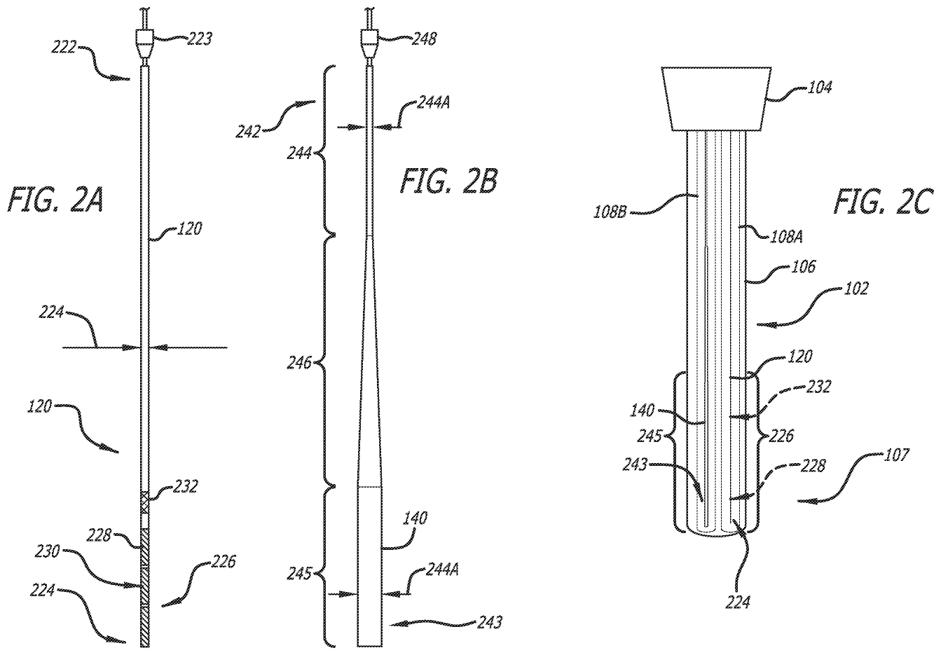

FIG. 2A illustrates a plan view of the primary stylet 120, in accordance with some embodiments. The primary stylet 120 defines a proximal end 222 and a distal end 224. In the illustrated embodiment, the distal end 224 may include a magnetic portion 226 having one or more magnets 228, where the magnets are configured to generate one or more magnetic fields to be detected in three dimensional space by a medical device tracking system. The U.S. published patent application No. 2013-0060116 titled INTEGRATED SYSTEM FOR INTRAVASCULAR PLACEMENT OF A CATHETER which shows and describes a stylet configured for magnetic tracking and a magnetic tracking system is included herein by reference in its entirety. In the illustrated embodiment, the magnetic portion 226 may include a sheath (or tubing) 230 extending along the magnetic region 226, and the one or more magnets 228 may be disposed within the sheath 230. In some embodiments, the sheath 230 may include polyimide tubing, PVC, silicon, or the like. In some embodiments, the magnetic portion 226 may define a weakened resistance to buckling.

Further in the illustrated embodiment, the distal end 224 of the primary stylet 120 may include one or more sensors 232 (e.g., electrodes) configured to detect one or more electrical signals (e.g., an ECG signal) during placement of the catheter 102. The U.S. patent No. 9,220,432 titled METHOD AND SYSTEM OF UTILIZING ECG SIGNAL FOR CENTRAL VENOUS CATHETER TIP POSITIONING which shows and describes a stylet having electrical sensors for detecting an ECG signal and a medical system for monitoring the ECG signal is included herein by reference in its entirety.

In some embodiments, the primary stylet 120 may include an optical fiber 236 extending along at least a portion of the primary stylet 120. The optical fiber 236 may be configured for shape sensing as taught by U.S. published patent application No. 2022-0034733 titled BRAGG GRATED FIBER OPTIC FLUCTUATION SENSING AND MONITORING SYSTEM showing and describing an optical fiber for shape sensing and a fiber optic shape sensing system which is included herein by reference in its entirety. By way of summary, the primary stylet 120 is generally configured tracking and/or placement confirmation during use. As such, the primary stylet 220 may include all or any subset of the magnetic region 226, the one or more sensors 232, or the optical fiber 236.

The primary stylet 120 defines a primary stylet diameter 224 extending between the proximal end 222 and the distal end 224 of the primary stylet 120. In the illustrated embodiment, the primary stylet diameter 224 is constant (i.e., the same) between the proximal end 222 and the distal end 224. In other embodiments, the primary stylet 120 may be tapered from the proximal end 222 to the distal end 224 with a larger stylet diameter 224 at the proximal end 222 and a smaller stylet diameter 224 or vice versa. In some embodiments, the primary stylet 120 may include a primary stylet attachment device 223 configured to selectively couple the primary stylet 120 to the first extension leg 112A so that longitudinal displacement of the primary stylet 120 with respect to the catheter 102 is prevented. In some embodiments, the primary stylet attachment device 223 may include a Luer locking capability configured to couple with a Luer connecting hub of the first extension leg 112A.

FIG. 2B illustrates a plan view of the secondary stylet 140, in accordance with some embodiments. As stated above, the secondary stylet 140 is generally configured to inhibit/prevent buckling of the primary stylet 120 and/or the catheter 102 during advancement of the catheter 102. More specifically, the secondary stylet 140 may be configured to inhibit/prevent buckling of the primary stylet 120 along the magnetic portion 226.

The secondary stylet 140 defines a proximal end 242 and a distal end 243. Similar to the primary stylet 120, the secondary stylet 140 may include a secondary stylet attachment device 248 configured to selectively couple the secondary stylet 140 to the second extension leg 112B so that longitudinal displacement of the secondary stylet 140 with respect to the catheter 102 is prevented. In some embodiments, the secondary stylet attachment device 248 may include a Luer locking capability configured to couple with a Luer connecting hub of the second extension leg 112B. Further, in some embodiments, although not shown, the secondary stylet 140 may, in addition to or in leu of primary stylet 120, include one or more of the magnetic region 226, the one or more sensors 232, or the optical fiber 236.

The secondary stylet 140 also a defines a column strength (i.e., resistance to buckling) along a length of the secondary stylet 140, where the column strength may vary along the length of the secondary stylet 140. In the illustrated embodiment, the varying column strength of the secondary stylet 140 is defined by a varying diameter of the secondary stylet 140 along its length. In other embodiments, the varying column strength may be defined varying properties of a secondary stylet material alone or in combination with the varying diameter of the secondary stylet 140. As such, in other embodiments, the diameter of the secondary stylet diameter 246 may be constant along its length.

The secondary stylet 140 may include (i) one or more tapered portions defining a change in diameter and (ii) one or more non-tapered portions defining a substantially constant diameter. In the illustrated embodiment, the secondary stylet 140 defines a proximal section 244, distal section 245, and a transitioning section 246 disposed between the proximal section 244 and the distal section 245. The proximal section 244 may include a proximal diameter 244A, where the proximal diameter 244A is substantially constant. The distal section 245 may include a distal diameter 245A, where the distal diameter 245A is substantially constant. The transitioning section 246 is configured to transition the proximal diameter 244A to the distal diameter 244A. As such, the transitioning section 246 defines a diameter that is between the proximal diameter 244A and the distal diameter 244A. In the illustrated embodiment, the transitioning section 246 includes a taper to transition the proximal diameter 244A to the distal diameter 244A. The distal diameter 245A is greater that the proximal diameter 244A.

In the illustrated embodiment, the proximal diameter 244A and the distal diameter 245A are configured to function in concert to prevent buckling of the distal section 245 (and, by association, the magnetic portion 226 of the primary stylet 120) during an instance of abutment as described above. The distal diameter 245A is configured to resist buckling when a longitudinal force along the distal section 245, as may be defined by a catheter insertion force, is below a distal column strength of the distal section 245 during use. As such, the distal column strength of the distal section 245 may define a catheter insertion force limit.

The proximal diameter 244A may be configured to prevent the longitudinal force from exceeding the distal column strength during use. More specifically, the proximal diameter 244A is configured to limit a longitudinal force that may be applied to secondary stylet 140. The proximal diameter 244A defines a proximal column strength of the proximal section 244. As such, the proximal diameter 244A may be configured to enable an external portion of the proximal section 244 (i.e., a portion of the of the proximal section 244 outside of the second extension leg lumen 112B) to buckle when an applied longitudinal force to the secondary stylet 140 exceeds the proximal column strength as further described below. By way of summary, the proximal column strength is less than the distal column strength such that a catheter insertion force applied to the secondary stylet 140 will cause the proximal section 244 to buckle, thereby preventing buckling of the distal section 245.

In some embodiments, the proximal ends 222, 242 may be constructed of a different material than the distal ends 224, 244. For example, the distal ends 224, 244 may be constructed of a first material, and the proximal section 222, 242 may be constructed of a second material, where the first material is more rigid than the second material. In some embodiments, the entire primary stylet 120 may be formed of a substantially rigid material. Conversely, portions of the primary stylet 120 may be formed of the rigid material, and other portions of the primary stylet 120 may be formed of a substantially flexible material. For example, the proximal end 222 may be formed of a more rigid material than the distal end 224. In similar fashion, in some embodiments, the entire secondary stylet 140 formed of a substantially rigid material or portions of the secondary stylet 140 may be formed of the rigid material and other portions of the secondary stylet 140 formed of a substantially flexible material. For example, the distal end 244 may be formed of a more rigid material than the proximal end 242.

FIG. 2C illustrates a cross sectional view of the catheter tube 106 including primary stylet 120 disposed within the first lumen 108A and the secondary stylet 140 disposed within the second lumen 108B, in accordance with some embodiments. In some embodiments, the primary stylet 120 and/or the secondary stylet 140 may be positioned along the catheter tube 106 such that the distal ends 224, 244 of the first and second stylets 120, 140, respectively are disposed adjacent the distal end 107 of the catheter tube 106.

As shown, the distal section 245 of the secondary stylet 140 extends along the magnetic portion 226 of the primary stylet 120. In the illustrated embodiment, a length of the distal section 245 may be the same or greater than a length of the magnetic portion 226. The transitioning section 246 or portion thereof may be disposed within the second lumen 108B. In some embodiments, a portion of the proximal section 244 may also be disposed within the second lumen 108B.

In some embodiments, the secondary stylet 140 may be optimized such that a single configuration of the secondary stylet 140 may be utilized for different catheter sizes and/or configurations. As such, optimizing the secondary stylet 140 for the different catheter configurations may normalize the tactile feel and force required to accurately place the catheter 102 and prevent buckling regardless of the catheter size. In some embodiments, optimizing the secondary stylet 140 for all catheter configurations will eliminate variations needed for insertion techniques for catheters 102 of different sizes. In other embodiments, the primary stylet 120 may be consistent (e.g., size and shape) and multiple configurations of the secondary stylet 140 may be provided to accommodate the different catheter configurations.

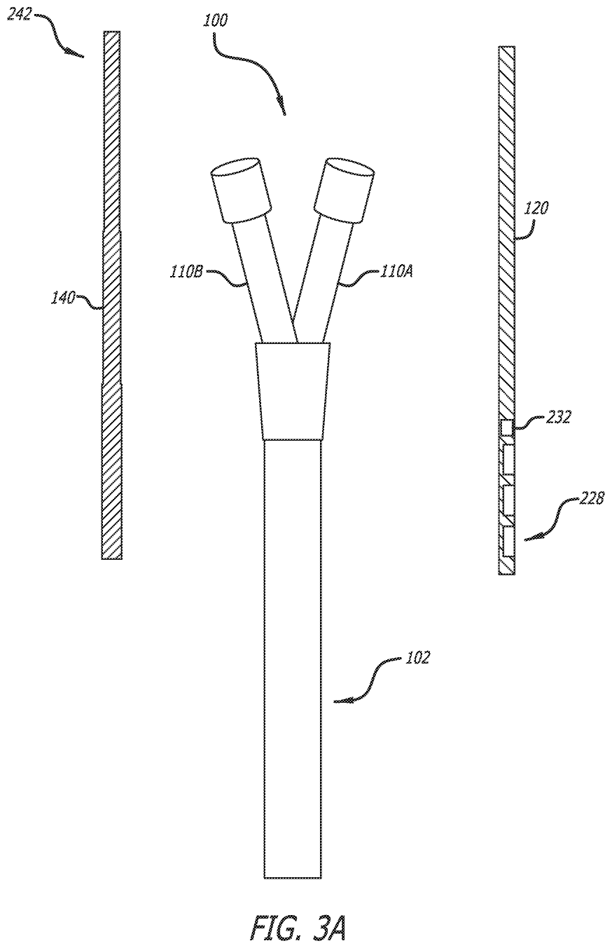

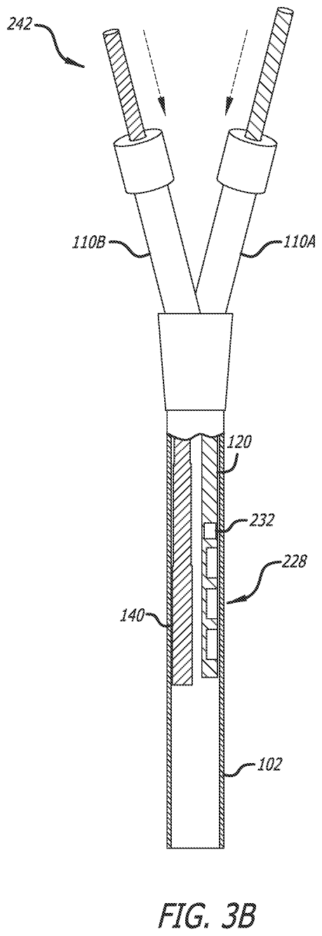

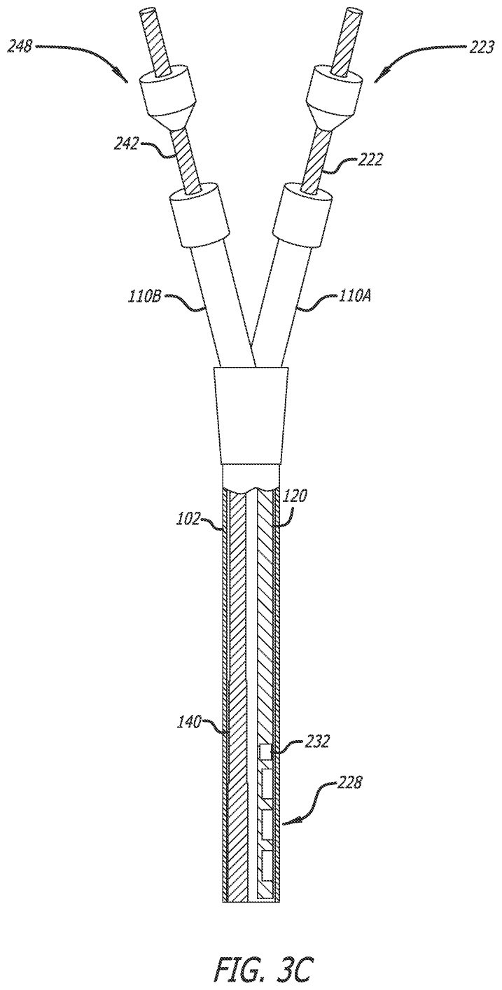

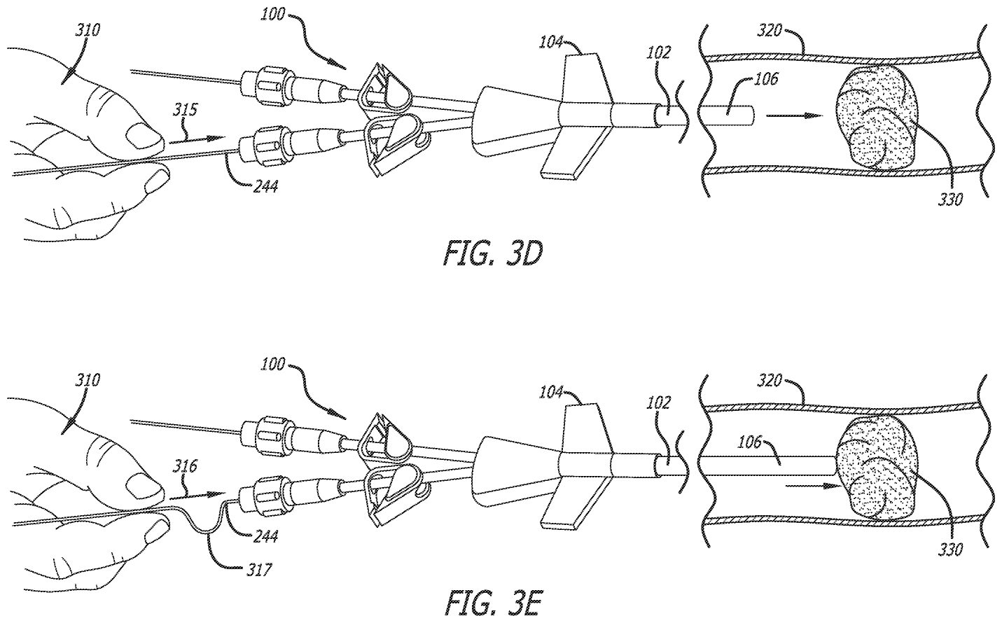

FIGS. 3A-3C illustrate various perspective views of the system 100 in accordance with an exemplary method of inserting a catheter 102. FIG. 3A illustrated an exposed view of the system 100. FIG. 3B illustrates the system 100 where the primary stylet 120 and the secondary stylet 140 are partially disposed with the catheter 102, and FIG. 3C illustrates the system 100 where the primary stylet 120 and the secondary stylet 140 are fully inserted with the catheter 102. In some embodiments, the catheter 102 may be prepared for insertion into the vasculature. As illustrated in FIG. 3A, the catheter insertion system 100 includes the secondary stylet 140 and the primary stylet 120 having the one or more sensors 232 configured to detect electrical signals and the one or more magnets 228. As illustrated in FIG. 3B, in some embodiments, the primary stylet 120 may be inserted into the first extension leg 110A. In some embodiments, the proximal end 222 of the primary stylet 120 may be selectively coupled to the proximal end of the catheter 102 to secure the location of the primary stylet 120 within the catheter 102. In some embodiments, the secondary stylet 140 may be inserted into the second extension leg 110B. In some embodiments, the proximal end 242 of the secondary stylet 140 may be selectively coupled to the proximal end of the catheter 102 to secure the location of the secondary stylet 140 within the catheter 102. In some embodiments, the primary stylet 120 or the secondary stylet 140 may be completely inserted into the catheter 102 before the other one of the primary stylet 120 or the secondary stylet 140 is inserted into the catheter 102. In some embodiments, the primary stylet 120 and the secondary stylet 140 may be simultaneously inserted into the catheter 102, as illustrated in FIG. 3B.

In some embodiments, the primary stylet attachment device 223 of the primary stylet 120 may be coupled between the primary stylet 120 and the first extension leg 110A. Similarly, the secondary stylet attachment device 248 of the secondary stylet 140 may be coupled between of the secondary stylet 140 and the second extension leg 110B. With the primary and secondary stylets 120, 140 disposed within the catheter 102, the catheter 102 may be inserted into the vasculature of the patient. In some embodiments, a medical device tracking system (not shown) may be utilized to detect and track the one or more magnets 228 on the primary stylet 120. Similarly, medical device tracking system may be utilized to confirm the location of the sensor 232 within the vasculature. Once the catheter 102 is placed within the vasculature of the patient, the primary stylet 120 and the secondary stylet 140 may be removed from catheter 102.

FIGS. 3D-3E illustrate the system 100 during advancement of the catheter 102 along the vasculature where an obstruction is present within the vasculature, in accordance with some embodiments. FIG. 3D illustrates advancement of the catheter 102 by the clinician prior to contact of the distal end 107 of the catheter 102 with the obstruction 330, and FIG. 3E illustrates the clinician applying the longitudinal force to the secondary stylet 140 after contact (abutment) of the distal end 107 of the catheter 102 with the obstruction 330. As shown in FIG. 3D, in some embodiments, the clinician 310 may grasp the secondary stylet 140 to apply a longitudinal force 315 to the proximal section 244 of the secondary stylet 140 to advance the catheter 102 along a blood vessel 320. As further shown in FIG. 3D, the proximal section 244 includes sufficient column strength to resist buckling of the proximal section 244 when the longitudinal force 315 is applied to advance the catheter 102 in the absence of an obstruction.

FIG. 3E illustrates the instance where the distal end 107 of the catheter 102 abuts the obstruction 330 halting advancement of the catheter 102. The clinician applies a longitudinal force 316 that is greater than the longitudinal force 315 of FIG. 3D in an effort to continue advancement of the catheter 102. The longitudinal force 316 exceeds the column strength of the proximal section 244 result in a buckling 317 of the proximal section 244 between the clinician 310 and the extension leg 110B. As such, the buckling 317 prevents a longitudinal force greater than the longitudinal force 316 from being applied to the secondary stylet 140 including the catheter 102 and the primary stylet 120. By way of summary, the bucking 317 of the proximal section 244 prevents buckling of the distal section 245, thereby protecting the magnetic portion 226 of the primary stylet 120, since the column strength of the distal section 245 is greater than the column strength of the proximal section 244.

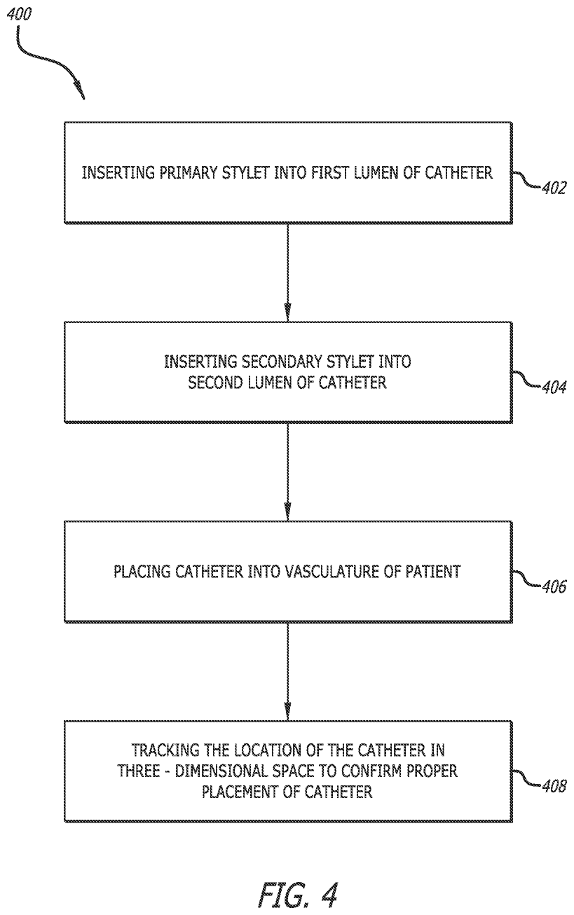

FIG. 4 illustrates a flow chart of an exemplary method 400 of placing the catheter 102 in the vasculature of a patient using the catheter insertion system 100, in accordance with some embodiments. In some embodiments, the method 400 may include inserting the primary stylet 120 into the catheter 102 (block 402). In some embodiments, the catheter 102 includes the hub 104 distally coupled to the catheter tube 106 having the first lumen 108A and the second lumen 108B therein. The hub 104 may be proximally coupled to the two or more extension legs 110A/110B wherein each extension leg 110A/110B is in fluid communication with one lumen 108A/108B. In some embodiments, inserting the primary stylet 120 into the catheter 102 includes slidably inserting the primary stylet 120 into the first lumen 108A. In some embodiments, the primary stylet 120 includes a magnetic region 226 and the one or more sensors 232. In some embodiments, the primary stylet 120 may have a constant primary stylet diameter 224 from the proximal end 222 to the distal end 224. In some embodiments, inserting the primary stylet 120 into the catheter 102 includes coupling the primary stylet attachment device 223 of the primary stylet 120 to the extension leg 110A.

The method 400 further includes inserting the secondary stylet 140 into the catheter 102 (block 404). In some embodiments, inserting the secondary stylet 140 into the catheter 102 includes slidably inserting the secondary stylet 140 into the second lumen 108B. In some embodiments, the secondary stylet 140 includes a proximal stylet diameter 244A that is less than a distal stylet diameter 245A. In some embodiments, inserting the secondary stylet 140 into the catheter 102 includes coupling the secondary stylet attachment device 248 of the secondary stylet 140 to the extension leg 110B.

The method 400 further includes placing the catheter 102 into the vasculature (block 406). In some embodiments, placing the catheter 102 into the vasculature includes placing the catheter 102 into the vasculature while having the primary stylet 120 and the secondary stylet 140 slidably inserted therein. In some embodiments, placing the catheter 102 includes slidably inserting the catheter 102 into the vasculature of the patient. In some embodiments, placing the catheter 102 includes grasping the secondary stylet 140 and applying a catheter insertion force to the secondary stylet 140 to insert the catheter 102. In some embodiments, placing the catheter 102 includes a applying a first catheter insertion force to the secondary stylet 140 and applying a second catheter insertion force, where the second catheter insertion force is greater than the first catheter insertion force. In such embodiments, the proximal section 244 resists buckling when the first catheter insertion force is applied and the proximal section 244 buckles when the second catheter insertion force is applied.

The method 400 further includes tracking the location of the catheter 102 in three-dimensional space to confirm proper placement of the catheter 102 (block 408). In some embodiments, tracking the location of the catheter 102 in three-dimensional space includes tracking the location of the one or more magnets 228 along the vasculature by a medical device tracking system. In some embodiments, tracking the location of the catheter 102 includes confirming the location of the one or more sensors 232 by a medical device tracking system detecting one or more electrical signals within the body. In some embodiments, once the location of the catheter 102 is confirmed, the primary stylet 120 and the secondary stylet 140 may be removed from the catheter 102.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter assembly, comprising:
a multi luminal catheter, comprising:
a first extension leg in fluid communication with a first lumen; and
a second extension leg in fluid communication with a second lumen
a primary stylet inserted within the first lumen, the primary stylet including a magnetic region configured to enable a medical tracking system to track the primary stylet during advancement of the catheter along a vasculature of a patient; and
a secondary stylet inserted within the second lumen,
wherein a distal section of the secondary stylet is configured to inhibit buckling of the magnetic region during advancement of the catheter assembly along a vasculature of a patient.

2. The assembly according to claim 1, wherein the distal section extends along the magnetic region.

3. The assembly according to claim 1, wherein the distal section defines a distal column strength greater than a column strength of the magnetic region of the primary stylet.

4. The assembly according to claim 3, wherein a proximal section of the secondary stylet defines a proximal column strength, the proximal column strength less than the distal column strength.

5. The assembly according to claim 4, wherein:
the proximal section defines a proximal diameter,
the distal section defines a distal diameter, and
the proximal diameter is less than the distal diameter.

6. The assembly according to claim 5, wherein:
the secondary stylet defines a transitioning section extending between the proximal section and the distal section, and
the transitioning section defines a diameter between distal diameter and the proximal diameter.

7. The assembly according to claim 6, wherein the transitioning section defines a taper extending along the transitioning section, the taper defining:
the proximal diameter at a proximal end of the transitioning section, and
the distal diameter at a distal end of the transitioning section.

8. The assembly according to claim 1, wherein the proximal section extends proximally beyond the second extension leg such that the proximal section is configured to receive a catheter insertion force applied thereto by a clinician.

9. The assembly according to claim 8, wherein the proximal column strength defines an insertion force limit for the catheter, when the catheter insertion force is applied to the secondary stylet.

10. The assembly according to claim 9, wherein the proximal section is configured to buckle when the catheter insertion force exceeds the insertion force limit.

11. The assembly according to claim 9, wherein the insertion force limit is configured to prevent buckling of the magnetic region.

12. The assembly according to claim 1, wherein:
the primary stylet includes a sheath extending along the magnetic region, and
the magnetic region includes one or more magnets disposed within the sheath.

13. The assembly according to claim 1, wherein the primary stylet includes one or more sensors configured to detect one or more electrical signals.

14. The assembly according to claim 13, wherein the one or more sensors include an electrode configured to detect an ECG signal to enable the medical tracking system to confirm a location of the catheter within the vasculature.

15. The assembly according to claim 1, wherein the primary stylet includes an optical fiber extending therealong, the optical fiber configured to enable shape sensing of the primary stylet by the medical tracking system.

16. The assembly according to claim 1, wherein the secondary stylet includes at least one of the magnetic region, one or more sensors configured to detect one or more electrical signals, or an optical fiber configured to enable shape sensing of the primary stylet by the medical tracking system.

17. The assembly according to claim 1, wherein the primary stylet includes a primary stylet attachment device configured to selectively couple the primary stylet to the first extension leg such that longitudinal displacement of the primary stylet with respect to the catheter is inhibited.

18. The assembly according to claim 1, wherein the secondary stylet includes a secondary stylet attachment device configured to selectively couple the secondary stylet to the second extension leg such that longitudinal displacement of the secondary stylet with respect to the catheter is inhibited.

19. A method for placing a catheter within a vasculature of a patient, comprising:
providing a catheter assembly, comprising:
multi-luminal catheter having a first extension leg in fluid communication with a first lumen and a second extension leg in fluid communication with a second lumen;
a primary stylet inserted within the first lumen, the primary stylet including a magnetic region configured to enable a medical tracking system to track of the primary stylet during advancement of the catheter along a vasculature of a patient; and
a secondary stylet inserted within the second lumen, the secondary stylet defining a proximal section extending proximally beyond the second extension leg; and
applying an insertion force to the proximal section to advance the catheter along the vasculature.

20. The method according to claim 19, wherein the secondary stylet defines a distal section having a distal column strength greater than a column strength of the magnetic region of the primary stylet.

21. The method according to claim 19, wherein providing the catheter assembly includes:
inserting the primary stylet into the first lumen; and
inserting a secondary stylet into the second lumen such that the distal section is disposed adjacent the magnetic region.

22. The method according to claim 20, wherein the proximal section includes a proximal column strength less than the distal column strength.

23. The method according to claim 22, wherein:
the proximal column strength defines an insertion force limit for the catheter, and
the proximal section is configured to buckle when the insertion force exceeds the insertion force limit.

24. The method according to claim 23, wherein the insertion force limit is configured to prevent buckling of the magnetic region.

25. The method according to claim 19, further comprising coupling a primary stylet attachment device between the primary stylet and a first extension leg of the catheter to inhibit longitudinal displacement of the primary stylet with respect to the catheter.

26. The method according to claim 19, further comprising coupling a secondary stylet attachment device between the secondary stylet and a second extension leg of the catheter to inhibit longitudinal displacement of the secondary stylet with respect to the catheter.
